(19) United States
(12) Patent Application Publication
(10) Pub. No.: US 2024/0117098 A1
(43) Pub. Date: Apr. 11, 2024

(54) BLOCK COPOLYMERS DERVIED FROM SHORT AND LONG ALKYL CHAIN ACRYLATES OR ACRYLAMIDES AND APPLICATIONS THEREOF

(71) Applicant: ISP Investments LLC, Wilmington, DE (US)

(72) Inventors: Rebecca Roisin Gibson, Sheffield, South Yorkshire (GB); ALAN FERNYHOUGH, Idle, Bradford, West Yorkshire (GB); OSAMA M. MUSA, Bedminster, NJ (US); Steven Peter ARMES

(73) Assignee: ISP Investments LLC, Wilmington, DE (US)

(21) Appl. No.: 18/255,023

(22) PCT Filed: Nov. 30, 2021
(86) PCT No.: PCT/US21/61211

§ 371 Date: May 30, 2023

(51) Int. Cl. C08F 293/00; A61K 8/81; A61Q 19/00; C08J 3/05; C09D 153/00

(52) U.S. Cl. CPC C08F 293/005; A61K 8/8164; A61Q 19/00; C08J 3/05; C09D 153/00; C08F 2438/03; C08J 2353/00

(57) ABSTRACT

The invention provides non-crosslinked and crosslinked block copolymers comprising at least one block A comprising repeating units derived from monomers comprising one or more C₁-C₃ alkyl (meth)acrylamides and/or (meth)acrylates and at least one block B comprising repeating units derived from monomers comprising one or more C₄-C₄₀ alkyl (meth)acrylamides. The invention further provides compositions comprising the block copolymers and applications thereof in various industrial areas including personal care. The invention furthermore provides compositions comprising colloidal particles of the block copolymers. The variables x, y, R₁, and R₂ are described herein.

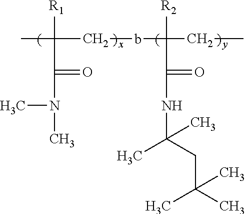

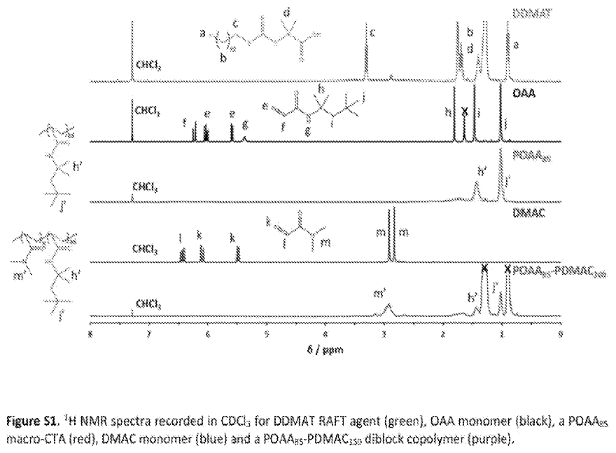

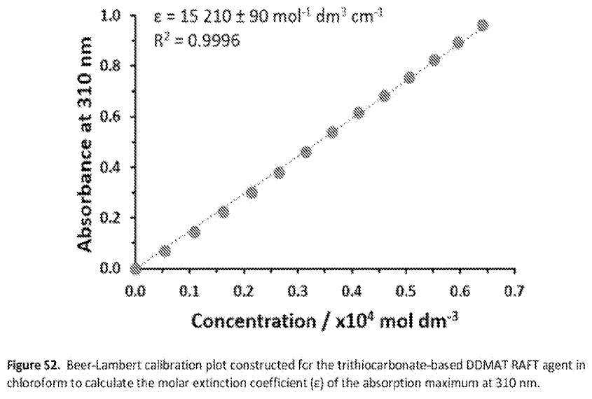

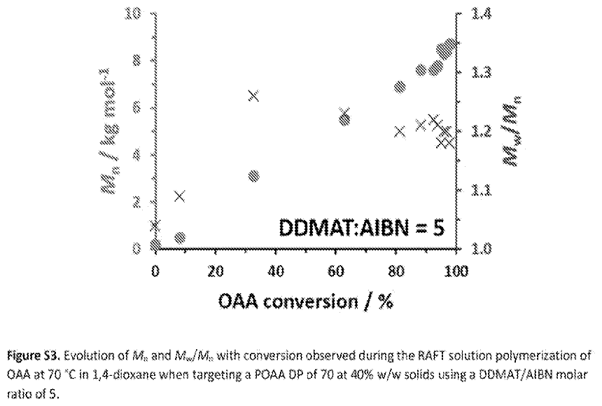

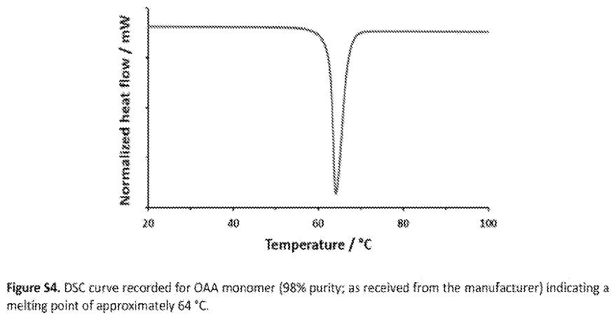

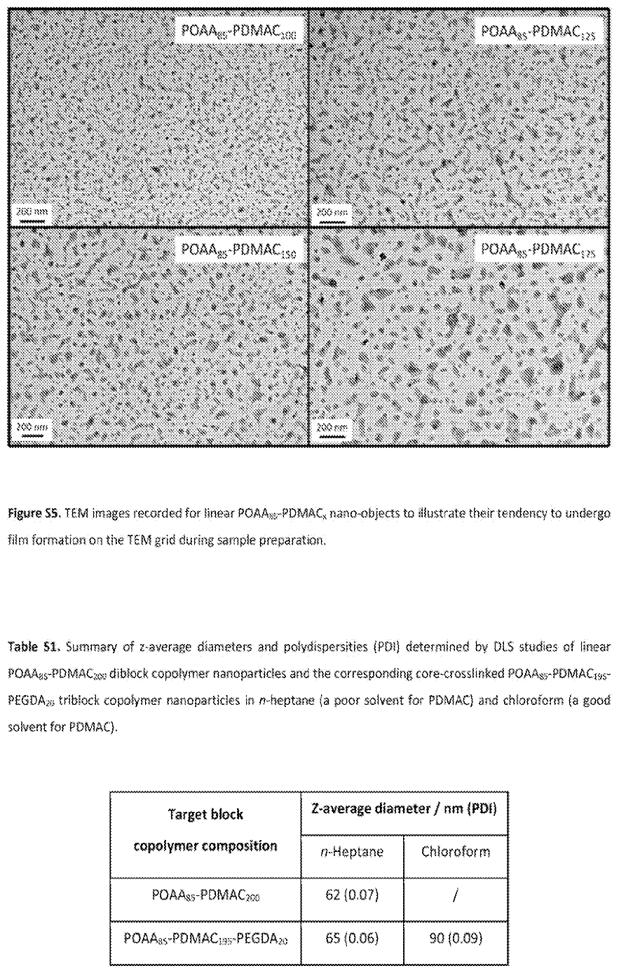

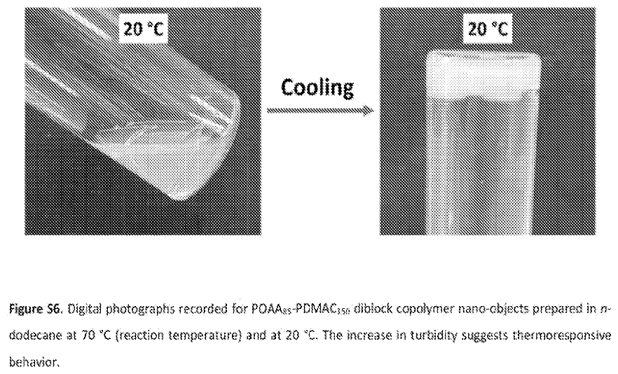

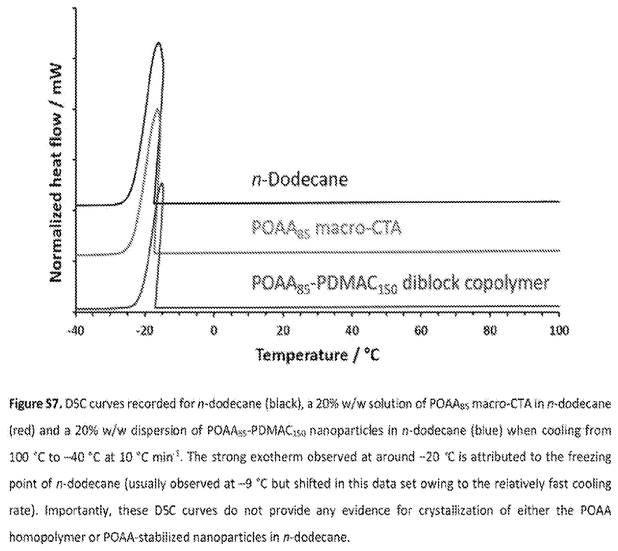

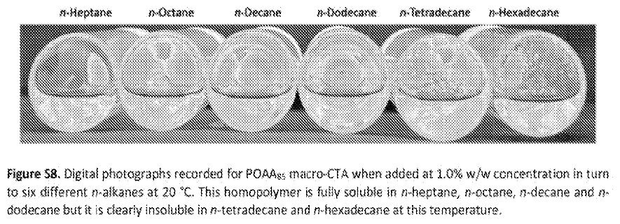

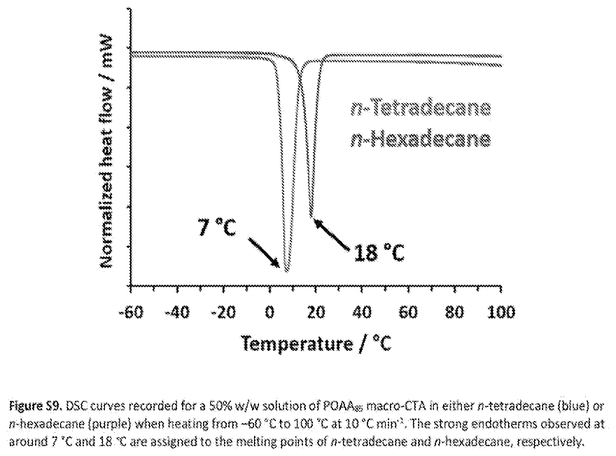

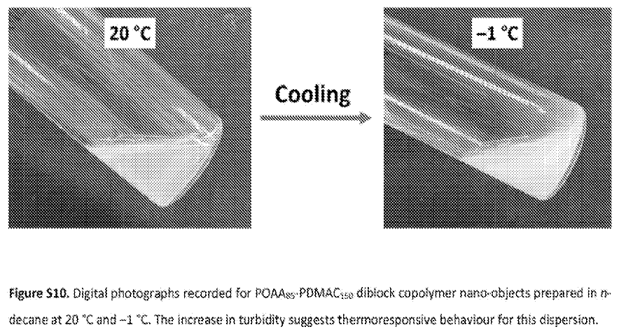

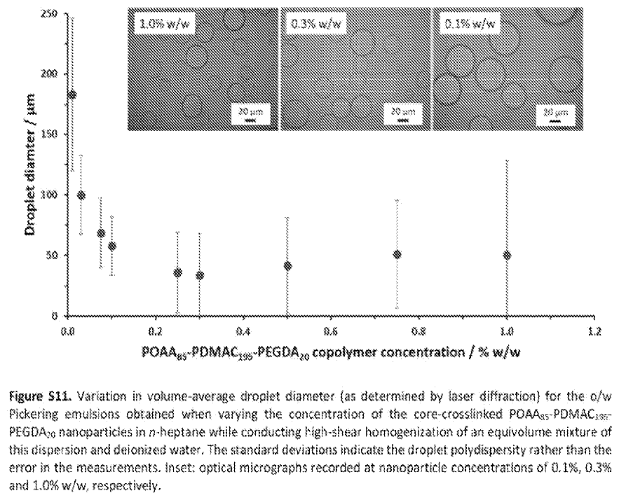

### BACKGROUND

### Field of the Invention

The disclosed and/or claimed inventive concept(s) provides non-crosslinked and crosslinked block copolymers derived by reversible addition-fragmentation chain transfer polymerization of a combination of short and long chain alkyl (meth)acrylates or (meth)acrylamides and industrial applications thereof.

### Description of Related Art

It is well-known that AB type of diblock copolymers undergo self-assembly both in the solid state and in solution. In the latter case, a diverse range of copolymer morphologies has been reported, including spheres, worms, or vesicles. Typically, the copolymer chains are first prepared in a non-selective solvent and then subjected to either a gradual change in solvency or a pH switch in a separate step, which is typically undertaken in dilute solution.

In recent years, polymerization-induced self-assembly (PISA) of diblock copolymers in a solvent that is selective for the growing second block has become increasingly popular. PISA offers two decisive advantages over traditional processing methods: (i) syntheses can be conducted at up to 50% w/w solids and (ii) diblock copolymer nanoparticles are obtained directly, without requiring any post-polymerization processing steps. When combined with PISA, controlled radical polymerization techniques such as atom transfer radical polymerization (ATRP) or reversible addition-fragmentation chain transfer (RAFT) polymerization have enabled the preparation of a wide range of well-defined nanoparticles. RAFT dispersion polymerization is known to allow the efficient synthesis of pure spherical, worm-like or vesicular morphologies in aqueous, alcoholic, or non-polar media as well as ionic liquids.

U.S. issued U.S. Pat. No. 5,194,549 discloses film-forming coating composition for application to a polypropylene substrate, which comprises a polypropylene-adherable polymer. In one of the embodiments, the polymer is prepared by radical polymerization of N-tertiary octyl acrylamide.

EP patent application 2,851,377 discloses a non-stop, one-pot method for the preparation of tri-block copolymers ABA by reversible addition fragmentation transfer (RAFT) polymerization using miniemulsion technology. The first ethylenically unsaturated monomer or first mixture of ethylenically unsaturated monomers comprise monomers selected from an exhaustive list of monomers that includes methacrylamide and the second ethylenically unsaturated monomer is selected from an exhaustive list of acrylate monomers that includes n-octyl methacrylate.

PCT published application 2012/144735 discloses a diblock copolymer that may facilitate formation of a finer nano pattern, and be used for manufacture of an electronic device including a nano pattern or a bio sensor, and the like, a method for preparing the same, and a method for forming a nano pattern using the same. The diblock copolymer comprises a hard segment including at least one specific acrylamide-based repeat unit, and a soft segment including at least one (meth)acrylate-based repeat unit.

Asada et al. in Polymer Journal. Volume 28, pages 145-149 (1996) describes surface-active properties of copolymers of N,N-dimethylacrylamide (DMA) with n-alkylsubstituted acrylamides, namely as n-butylacrylamide (NBA), n-hexylacrylamide (NHA), n-octylacrylamide (NOA), n-dodecylacrylamide (NDA), and their adsorption onto polystyrene (PS) latex particles.

Kulai et al. in ACS Macro Letters, Volume 4, pages 809-813 (2015) describes a new range of tin-based reversible addition-fragmentation chain-transfer (RAFT) agents is described and evaluated for the polymerization of acrylamides, methyl acrylate and styrene. To evaluate the ability of the novel RAFT agents to induce RDRP, the authors polymerized a selection of more-activated monomers, namely, N-ispropropylacrylamide (NIPAM), N-tert-octyl acrylamide (TOA), methyl acrylate (MA), and styrene (St), using Sn-RAFT agents at 60° C. with AIBN as thermal initiator.

It has been found that block copolymers derived by reversible addition-fragmentation chain transfer polymerization of a combination of short and long chain alkyl (meth)acrylates or (meth)acrylamides have unique properties that lend them to potential applications across multiple industries.

### SUMMARY

In a first aspect, the disclosed and/or claimed inventive concept(s) provides a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; and at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons.

In a second aspect, the disclosed and/or claimed inventive concept(s) provides a crosslinked block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and repeating units derived from at least one crosslinking agent.

In a third aspect, the disclosed and/or claimed inventive concept(s) provides a composition comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and optionally, repeating units derived from at least one crosslinking agent.

In a fourth aspect, the disclosed and/or claimed inventive concept(s) provides a personal care composition comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and optionally, repeating units derived from at least one crosslinking agent.

In a fifth aspect, the disclosed and/or claimed inventive concept(s) provides a composition in the form of colloidal particles comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and optionally, repeating units derived from at least one crosslinking agent.

In a sixth aspect, the disclosed and/or claimed inventive concept(s) provides a Pickering emulsion composition comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and optionally, repeating units derived from at least one crosslinking agent.

### DETAILED DESCRIPTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

All patents, published patent applications, and non-patent publications referenced in any portion of this application are herein expressly incorporated by reference in their entirety to the same extent as if each individual patent or publication was specifically and individually indicated to be incorporated by reference.

All articles and/or methods disclosed herein can be made and executed without undue experimentation based on the present disclosure. While the articles and methods of the disclosed and/or claimed inventive concept(s) have been described in terms of aspects, it will be apparent to those of ordinary skill in the art that variations may be applied to the articles and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope, and concept of the disclosed and/or claimed inventive concept(s).

As utilized in accordance with the disclosure, the following terms, unless otherwise indicated, shall be understood to have the following meanings.

The use of the word “a” or “an” when used in conjunction with the term “comprising” may mean “one,” but it is also consistent with the meaning of “one or more,” “at least one,” and “one or more than one.” The use of the term “or” is used to mean “and/or” unless explicitly indicated to refer to alternatives only if the alternatives are mutually exclusive, although the disclosure supports a definition that refers to only alternatives and “and/or.”

Throughout this application, the term “about” is used to indicate that a value includes the inherent variation of error for the quantifying device, the method being employed to determine the value, or the variation that exists among the study subjects. For example, but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first”, “second”, “third”, “fourth”, etc.) is solely for the purpose of differentiating between two or more items and, unless otherwise stated, is not meant to imply any sequence or order or importance to one item over another or any order of addition.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “or combinations thereof” as used herein refers to all permutations and combinations of the listed items preceding the term. For example, “A, B_{Xn}, B_{Xn+1}, or combinations thereof” is intended to include at least one of: A, B_{Xn}, B_{Xn+1}, AB_{Xn}, A B_{Xn+1}, B_{Xn}B_{Xn+1}, or AB_{Xn}B_{Xn+1} and, if order is important in a particular context, also B_{Xn}A, B_{Xn+1}A, B_{Xn+1}B_{Xn}, B_{Xn+1}B_{Xn}A, B_{Xn}B_{Xn+1}A, AB_{Xn+1}B_{Xn}, B_{Xn}AB_{Xn+1}, or B_{Xn+1}AB_{Xn}. Continuing with this example, expressly included are combinations that contain repeats of one or more item or term, such as B_{Xn}B_{Xn}, AAA, MB_{Xn}, B_{Xn}B_{Xn}B_{Xn+1}, AAAB_{Xn}B_{Xn+1}B_{Xn+1}B_{Xn+1}B_{Xn+1}, B_{Xn+1}B_{Xn}B_{Xn}AAA, B_{Xn+1}A B_{Xn}AB_{Xn}B_{Xn}, and so forth. The skilled artisan will understand that typically there is no limit on the number of items or terms in any combination, unless otherwise apparent from the context.

The term “each independently selected from the group consisting of” means when a group appears more than once in a structure, that group may be selected independently each time it appears.

The term “hydrocarbyl” includes straight-chain and branched-chain alkyl, alkenyl, alkynyl, cycloalkyl, cycloalkenyl, aryl groups, and combinations thereof with optional heteroatom(s). A hydrocarbyl group may be mono-, di- or polyvalent.

The term “alkyl” refers to a functionalized or unfunctionalized, monovalent, straight-chain, branched-chain, or cyclic C₁-C₆₀ hydrocarbyl group optionally having one or more heteroatoms. In one non-limiting embodiment, an alkyl is a C₁-C₄₅ hydrocarbyl group. In another non-limiting embodiment, an alkyl is a C_{1-C30} hydrocarbyl group. Non-limiting examples of alkyl include methyl, ethyl, n-propyl, isopropyl, n-butyl, isobutyl, tert-butyl, n-pentyl, isopentyl, n-hexyl, n-heptyl, n-octyl, 2-ethylhexyl, tert-octyl, iso-norbornyl, n-dodecyl, tert-dodecyl, n-tetradecyl, n-hexadecyl, n-octadecyl, n-eicosyl, cyclobutyl, cyclopentyl, cyclohexyl, and the like. The definition of “alkyl” also includes groups obtained by combinations of straight-chain, branched-chain and/or cyclic structures.

The term “aryl” refers to a functionalized or unfunctionalized, monovalent, aromatic hydrocarbyl group optionally having one or more heteroatoms. The definition of aryl includes carbocyclic and heterocyclic aromatic groups. Non-limiting examples of aryl groups include phenyl, naphthyl, indenyl, indanyl, azulenyl, fluorenyl, anthracenyl, furyl, thienyl, pyridyl, pyrrolyl, oxazolyl, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinyl, pteridinyl, carbazolyl, acridinyl, phenazinyl, phenothiazinyl, phenoxyazinyl, pyrazolo[1,5-c]triazinyl, and the like.

The term “aralkyl” refers to an alkyl group comprising one or more aryl substituent(s) wherein “aryl” and “alkyl” are as defined above. Non-limiting examples of aralkyl groups include benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like.

The term “alkylene” refers to a functionalized or unfunctionalized, divalent, straight-chain, branched-chain, or cyclic C₁-C₁₀ hydrocarbyl group optionally having one or more heteroatoms. In one non-limiting embodiment, an alkylene is a C₁-C₃₀ group. In another non-limiting embodiment, an alkylene is a C₁-C₂₀ group. Non-limiting examples of alkylene groups include:

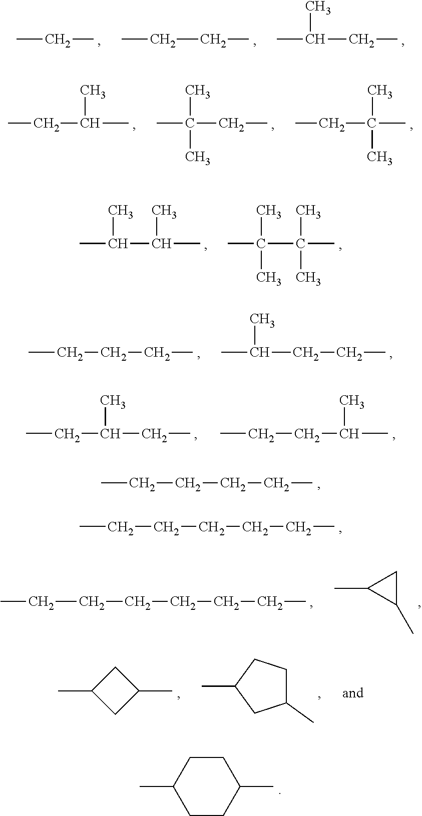

The term “arylene” refers to a functionalized or unfunctionalized, divalent, aromatic hydrocarbyl group optionally having one or more heteroatoms. The definition of arylene includes carbocyclic and heterocyclic groups. Non-limiting examples of arylene groups include phenylene, naphthylene, pyridinylene, and the like.

The term “heteroatom” refers to oxygen, nitrogen, sulfur, silicon, phosphorous, or halogen. The heteroatom(s) may be present as a part of one or more heteroatom-containing functional groups. Non-limiting examples of heteroatom-containing functional groups include ether, hydroxy, epoxy, carbonyl, carboxamide, carboxylic ester, carboxylic acid, imine, imide, amine, sulfonic, sulfonamide, phosphonic, and silane groups. The heteroatom(s) may also be present as a part of a ring such as in heteroaryl and heteroarylene groups.

The term “halogen” or “halo” refers to Cl, Br, I, or F.

The term “ammonium” includes protonated NH₃ and protonated primary, secondary, and tertiary organic amines.

The term “functionalized” with reference to any moiety refers to the presence of one or more functional groups in the moiety. Various functional groups may be introduced in a moiety by way of one or more functionalization reactions known to a person having ordinary skill in the art. Non-limiting examples of functionalization reactions include: alkylation, epoxidation, sulfonation, hydrolysis, amidation, esterification, hydroxylation, dihydroxylation, amination, ammonolysis, acylation, nitration, oxidation, dehydration, elimination, hydration, dehydrogenation, hydrogenation, acetalization, halogenation, dehydrohalogenation, Michael addition, aldol condensation, Canizzaro reaction, Mannich reaction, Clasien condensation, Suzuki coupling, and the like. In one non-limiting embodiment, the term “functionalized” with reference to any moiety refers to the presence of one more functional groups selected from the group consisting of alkyl, alkenyl, hydroxyl, carboxyl, halogen, alkoxy, amino, imino, and combinations thereof, in the moiety.

The term “monomer” refers to a small molecule that chemically bonds during polymerization to one or more monomers of the same or different kind to form a polymer.

The term “polymer” refers to a large molecule comprising one or more types of monomer residues (repeating units) connected by covalent chemical bonds. By this definition, polymer encompasses compounds wherein the number of monomer units may range from very few, which more commonly may be called as oligomers, to very many. Non-limiting examples of polymers include homopolymers, and non-homopolymers such as copolymers, terpolymers, tetrapolymers and the higher analogues. The polymer may have a random, block, and/or alternating architecture.

The term “homopolymer” refers to a polymer that consists essentially of a single monomer type.

The term “non-homopolymer” refers to a polymer that comprises more than one monomer types.

The term “copolymer” refers to a non-homopolymer that comprises two different monomer types.

The term “terpolymer” refers to a non-homopolymer that comprises three different monomer types.

The term “branched” refers to any non-linear molecular structure. The term includes both branched and hyper-branched structures.

The term “block copolymer” refers to a polymer comprising at least two blocks of polymerized monomers. Any block may be derived from either a single monomer resulting in a homopolymeric subunit, or two or more monomers resulting in a copolymeric (or non-homopolymeric) subunit in the block copolymer. The block copolymers may be diblock copolymers (i.e., polymers comprising two blocks of monomers), triblock copolymers (i.e., polymers comprising three blocks of monomers), multiblock copolymers (i.e., polymers comprising more than three blocks of monomers), and combinations thereof. The block copolymers may be linear, branched, star or comb like, and have structures such as [A][B], [A][B][A], [A][B][C], [A][B][A][B], [A][B][C][B], etc. An exemplary representation of block copolymer is [A]ₓ[B]_{y} or [A]ₓ[B]_{y}[C]_{z}, wherein x, y and z are the degrees of polymerization (DP) of the corresponding blocks [A], [B], and [C]. Additional insight into the chemistry, characterization and applications of block copolymers may be found in the book ‘Block Copolymers: Synthetic Strategies, Physical Properties, and Applications’, by Nikos Hadjichristidis, Stergios Pispas, and George Floudas, John Wiley and Sons (2003), the contents of which are herein incorporated in its entirety by reference.

The term “controlled radical polymerization” refers to a specific radical polymerization process, also denoted by the term of “living radical polymerization”, in which use is made of control agents, such that the block copolymer chains being formed are functionalized by end groups capable of being reactivated in the form of free radicals by virtue of reversible transfer or reversible termination reactions.

The term “addition-fragmentation” refers to a two-step chain transfer mechanism during polymerization leading to homopolymers and block copolymers wherein a radical addition is followed by fragmentation to generate a new radical species.

The term RAFT refers to reversible addition-fragmentation chain transfer.

The term “free radical addition polymerization initiator” refers to a compound used in a catalytic amount to initiate a free radical addition polymerization. The choice of initiator depends mainly upon its solubility and its decomposition temperature.

The term “alkyl acrylate” refers to an alkyl ester of an acrylic acid or an alkyl acrylic acid.

The term “alkyl acrylamide” refers to an alkyl amide of an acrylic acid or an alkyl acrylic acid.

The term “moiety” refers to a part or a functional group of a molecule.

In the block copolymer structures, the notation -b- on the polymer backbone is meant to denote block configuration of repeating units. An exemplary block copolymer structure is shown below:

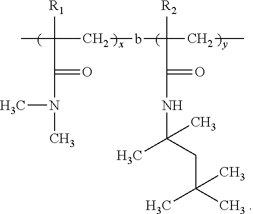

The terms “personal care composition” and “cosmetics” refer to compositions intended for use on or in the human body, such as skin, sun, hair, oral, cosmetic, and preservative compositions, including those to alter the color and appearance of skin and hair.

The term “pharmaceutical composition” refers to any composition comprising at least one pharmaceutically active ingredient, as well as any product which results, directly or indirectly, from combination, complexation, or aggregation of any two or more of the ingredients, from dissociation of one or more of the ingredients, or from other types of reactions or interactions of one or more of the ingredients.

The term “coating composition” refers to an aqueous-based or solvent-based liquid composition that may be applied to a substrate and thereafter solidified (for example, by radiation, air curing, post-crosslinking or ambient temperature drying) to form a hardened coating on the substrate.

The term “Pickering emulsion” refers to an emulsion of any type, either oil-in-water (o/w), water-in-oil (w/o), or multiple emulsion, stabilized by the presence of nanometric or micrometric solid particles at the interface between the different phases.

The term “colloidal” refers to the state of matter having nanometer dimensions.

The term “oilfield composition” refers to a composition that may be used in the exploration, extraction, recovery, and/or completion of any hydrocarbon. Non-limiting examples of oilfield compositions include drilling fluids, cementing fluids, anti-agglomerants, kinetic hydrate inhibitors, shale swelling inhibitors, drilling muds, servicing fluids, gravel packing muds, friction reducers, fracturing fluids, completion fluids, and work over fluids.

All percentages, ratio, and proportions used herein are based on a weight basis unless other specified.

In a first aspect, the disclosed and/or claimed inventive concept(s) provides a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; and at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons.

In one non-limiting embodiment, the block copolymer according to the disclosed and/or claimed inventive concept(s) comprises at least one block A comprising repeating units derived from at least one monomer selected from the group consisting of functionalized or unfunctionalized acylamide, N-methyl acrylamide, N,N′-dimethyl acrylamide, (meth)acylamide, N-methyl (meth)acrylamide, N,N′-dimethyl (meth)acrylamide, and combinations thereof; and at least one block B comprising repeating units derived from at least one monomer selected from the group consisting of functionalized or unfunctionalized N-butyl acrylamide, N-sec-butyl acrylamide, N-tert-butyl acrylamide, N-isobutyl acrylamide, N-neopentyl acrylamide, N-hexyl acrylamide, N-cyclohexyl acrylamide, N-2-ethylbutyl acrylamide, N-heptyl acrylamide, N-2-heptyl acrylamide, N-2-ethylhexyl acrylamide, N-octyl acrylamide, N-tert-octyl acrylamide, N-iso-decyl acrylamide, N-dodecyl acrylamide, N-lauryl acrylamide, N-2-propylheptyl acrylamide, N-behenyl acrylamide, N-tetradecyl acrylamide, N-octadecyl acrylamide, N-butyl (meth)acrylamide, N-sec-butyl (meth)acrylamide, N-tert-butyl (meth)acrylamide, N-isobutyl (meth)acrylamide, N-neopentyl (meth)acrylamide, N-hexyl (meth)acrylamide, N-cyclohexyl (meth)acrylamide, N-2-ethylbutyl (meth)acrylamide, N-heptyl (meth)acrylamide, N-2-heptyl (meth)acrylamide, N-2-ethylhexyl (meth)acrylamide, N-octyl (meth)acrylamide, N-tert-octyl (meth)acrylamide, N-iso-decyl (meth)acrylamide, N-dodecyl (meth)acrylamide, N-lauryl (meth)acrylamide, N-2-propylheptyl (meth)acrylamide, N-behenyl (meth)acrylamide, N-tetradecyl (meth)acrylamide, N-octadecyl (meth)acrylamide, and combinations thereof.

In one non-limiting embodiment, the block copolymer according to the disclosed and/or claimed inventive concept(s) is a diblock, triblock, or multiblock copolymer.

In one non-limiting embodiment, the crosslinked block copolymer according to the disclosed and/or claimed inventive concept(s) is a diblock, triblock, or multiblock crosslinked copolymer.

In a second aspect, the disclosed and/or claimed inventive concept(s) provides a crosslinked block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and repeating units derived from at least one crosslinking agent.

In one non-limiting embodiment, the crosslinking agent is selected from the group consisting of allyl acrylate, ethylene glycol acrylate, ethylene glycol diacrylate, diethylene glycol acrylate, diethylene glycol diacrylate, triethylene glycol acrylate, triethylene glycol diacrylate, polyethylene glycol acrylate, polyethylene glycol diacrylate, butylene glycol acrylate, butylene glycol diacrylate, glycidyl acrylate, N-hydroxymethyl acrylamide, N,N′-methylene diacrylamide, trimethylolpropane triacrylate, allyl methacrylate, ethylene glycol methacrylate, ethylene glycol dimethacrylate, diethylene glycol methacrylate, diethylene glycol dimethacrylate, triethylene glycol methacrylate, triethylene glycol dimethacrylate, polyethylene glycol methacrylate, polyethylene glycol dimethacrylate, butylene glycol methacrylate, butylene glycol dimethacrylate, glycidyl methacrylate, N-hydroxymethyl methacrylamide, N,N′-methylene dimethacrylamide, trimethylolpropane trimethacrylate, divinylbenzene, triallyl isocyanurate, triallyl citrate, diethyleneglycol divinyl ether, pentaerythritol triallyl ether, pentaerythritol triacrylate, pentaerythritol trimethacrylate, pentaerythritol tetraacrylate, pentaerythritol tetramethacrylate, methylene bisacrylamide, methylene bismethacrylamide, N,N′-divinylimidazolidone, triallyl-1,3,5-triazine-2,4,6(1H,3H,5H)-trione, 2,4,6-triallyloxy-1,3,5-triazine, and combinations thereof.

In one non-limiting embodiment, the block copolymer according to the disclosed and/or claimed inventive concept(s) has a structure selected from the group consisting of

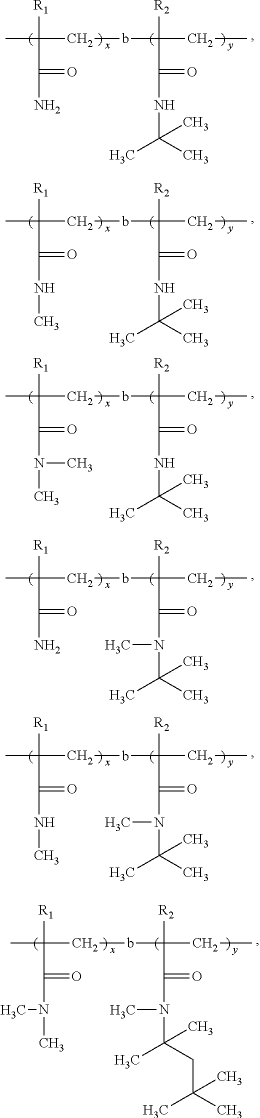

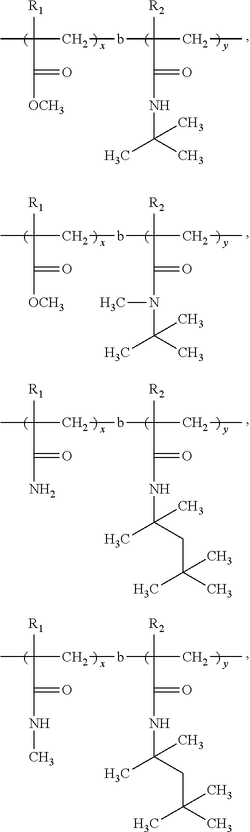

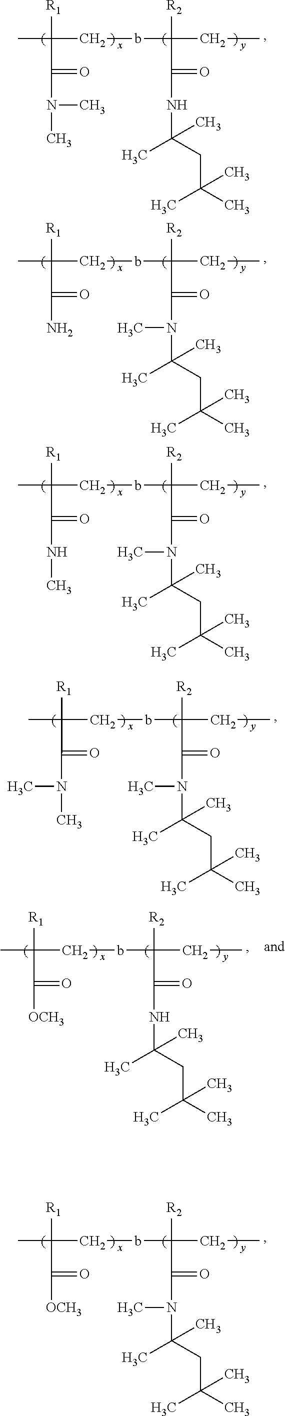

wherein each x and y is independently an integer having a value from about 10 to about 10000, and each R₁ and R₂ is independently selected from the group consisting of hydrogen, methyl, and combinations thereof.

In a third aspect, the disclosed and/or claimed inventive concept(s) provides a composition comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and optionally, repeating units derived from at least one crosslinking agent.

In one non-limiting embodiment, the composition is a personal care composition, pharmaceutical composition, coating composition, construction composition, nutritional composition, agricultural composition, adhesive composition, oilfield composition, household, industrial and institutional composition, cementing fluid, servicing fluid, gravel packing mud, fracturing fluid, completion fluid, work-over fluid, spacer fluid, drilling mud, biocide, ink, paper, polish, membrane, metal working fluid, plastic, textile, printing composition, lubricant, detergent, battery composition, glass coating composition, or preservative composition.

In a fourth aspect, the disclosed and/or claimed inventive concept(s) provides a personal care composition comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and optionally, repeating units derived from at least one crosslinking agent.

In one non-limiting embodiment, the personal care composition is selected from the group consisting of a sun care composition, a face care composition, a lip care composition, an eye care composition, a skin care composition, an after-sun composition, a body care composition, a nail care composition, an anti-aging composition, an insect repellant, an oral care composition, a deodorant composition, a hair care composition, a conditioning composition, a color cosmetic composition, a color-protection composition, a self-tanning composition, or a foot care composition.

In one non-limiting embodiment, the personal care composition further comprises at least one additive selected from the group consisting of UV actives, UV active solubilizers, oils, waxes, solvents, emulsifiers, preservatives, antioxidants, antiradical protecting agents, vitamins, perfumes, insect repellants, dyes, pigments, humectants, fillers, thickeners, film formers, stabilizers, buffers, spreading agents, pearlizing agents, electrolytes, acids, bases, crystalline structuring agents, abrasives, pharmaceutically or cosmetically acceptable excipients, and combinations thereof.

In a fifth aspect, the disclosed and/or claimed inventive concept(s) provides a composition in the form of colloidal particles comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and optionally, repeating units derived from at least one crosslinking agent.

In one non-limiting embodiment, the colloidal particles have spherical morphologies. In another non-limiting embodiment, the colloidal particles have non-spherical morphologies. Non-limiting examples of colloidal particles having non-spherical morphologies include worms and vesicles. Further insight into the structure and properties of colloidal particles having non-spherical morphologies may be found in the publication J. Am. Chem. Soc., 2014, volume 136, 10174-10185, the contents of which are herein incorporated in its entirety by reference.

In one non-limiting embodiment, the colloidal particles have a mean diameter ranging from about 10 nanometers to about 1000 nanometers, as measured by a suitable technique such as, for example, Dynamic Light Scattering. In another non-limiting embodiment, the colloidal particles have a mean diameter ranging from about 10 nanometers to about 500 nanometers. In yet another non-limiting embodiment, the colloidal particles have a mean diameter ranging from about 10 nanometers to about 250 nanometers. In yet another non-limiting embodiment, the colloidal particles have a mean diameter ranging from about 10 nanometers to about 100 nanometers.

In a sixth aspect, the disclosed and/or claimed inventive concept(s) provides a Pickering emulsion composition comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each alkyl moiety independently has from 4 to about 40 carbons; and optionally, repeating units derived from at least one crosslinking agent.

In one non-limiting embodiment, the Pickering emulsion composition comprises a block copolymer comprising at least one block A comprising repeating units derived from at least one monomer selected from the group consisting of functionalized or unfunctionalized acylamide, N-methyl acrylamide, N,N′-dimethyl acrylamide, (meth)acylamide, N-methyl (meth)acrylamide, N,N′-dimethyl (meth)acrylamide, and combinations thereof; and at least one block B comprising repeating units derived from at least one monomer selected from the group consisting of functionalized or unfunctionalized N-butyl acrylamide, N-sec-butyl acrylamide, N-tert-butyl acrylamide, N-isobutyl acrylamide, N-neopentyl acrylamide, N-hexyl acrylamide, N-cyclohexyl acrylamide, N-2-ethylbutyl acrylamide, N-heptyl acrylamide, N-2-heptyl acrylamide, N-2-ethylhexyl acrylamide, N-octyl acrylamide, N-tert-octyl acrylamide, N-iso-decyl acrylamide, N-dodecyl acrylamide, N-lauryl acrylamide, N-2-propylheptyl acrylamide, N-behenyl acrylamide, N-tetradecyl acrylamide, N-octadecyl acrylamide, N-butyl (meth)acrylamide, N-sec-butyl (meth)acrylamide, N-tert-butyl (meth)acrylamide, N-isobutyl (meth)acrylamide, N-neopentyl (meth)acrylamide, N-hexyl (meth)acrylamide, N-cyclohexyl (meth)acrylamide, N-2-ethylbutyl (meth)acrylamide, N-heptyl (meth)acrylamide, N-2-heptyl (meth)acrylamide, N-2-ethylhexyl (meth)acrylamide, N-octyl (meth)acrylamide, N-tert-octyl (meth)acrylamide, N-iso-decyl (meth)acrylamide, N-dodecyl (meth)acrylamide, N-lauryl (meth)acrylamide, N-2-propylheptyl (meth)acrylamide, N-behenyl (meth)acrylamide, N-tetradecyl (meth)acrylamide, N-octadecyl (meth)acrylamide, and combinations thereof.

Pickering emulsions are emulsions of any type, either oil-in-water (o/w), water-in-oil (w/o), or even multiple, stabilized by solid particles in place of surfactants. Pickering emulsions retain the basic properties of classical emulsions stabilized by surfactants (emulsifiers), so that a Pickering emulsion can be substituted for a classical emulsion in most applications of emulsions. The stabilization by solid particles brings about specific properties to such emulsions. The high resistance to coalescence is a major benefit of the stabilization by solid particles. The ‘surfactant-free’ character makes them attractive to several applications fields, non-limiting examples of which include cosmetic and pharmaceutical applications where surfactants often show adverse effects. Solid stabilizing particles are necessarily smaller than emulsion droplets. Solid particles of nanometric size (or sub-micron, ˜100 nanometers) allow the stabilization of droplets as small as few micrometers diameter, stabilization of larger droplets is possible as well. Micron-sized solid particles can stabilize larger droplets, the diameter of which possibly reaching few millimeters. The availability of stable millimeter-sized emulsions is a supplementary benefit of Pickering emulsions with respect to classical emulsions; this possibility comes from their high stability against coalescence.

Properties and applications of Pickering emulsions in general are described by Chevalier and Bolzinger in Colloids and Surfaces A: Physicochemical and Engineering Aspects, 2013, volume 439, 23-34, the contents of which are herein incorporated in its entirety by reference.

In one non-limiting embodiment, the non-crosslinked or crosslinked block copolymer that is a component of Pickering emulsion compositions according to the disclosed and/or claimed inventive concept(s), is present in an amount from about 0.01% by weight to about 20% by weight of the composition. In another non-limiting embodiment, the block copolymer is present in an amount from about 0.1% by weight to about 10% by weight of the composition. In yet another non-limiting embodiment, the block copolymer is present in an amount from about 0.25% by weight to about 5.0% by weight of the composition.

In one non-limiting embodiment, the block copolymer is present in the form of colloidal particles in the Pickering emulsion composition. In another non-limiting embodiment, the block copolymer is present in the form of spherical colloidal particles in the Pickering emulsion composition.

In one non-limiting embodiment, the composition comprising the non-crosslinked or crosslinked block copolymers according to disclosed and/or claimed inventive concepts is a hydrogel. Further insights into the properties and applications of hydrogels may be found in the review article by Ullah and coworkers in Materials Science and Engineering C, 2015, volume 57, 414-433 the contents of which are herein incorporated in its entirety by reference.

Additional insight into compositional functionality and application(s) of the non-crosslinked or crosslinked block copolymers according to the disclosed and/or claimed inventive concepts is disclosed in Gibson et. al, Polymer Chemistry, 2021, Volume 12, 2165-2174, the disclosure of which is herein incorporated by reference in its entirety.

### Methods of Synthesis

RAFT polymerization is one of the most robust and versatile methods for providing living characteristics to radical polymerization. With appropriate selection of the RAFT agent for the monomers and reaction conditions, it is applicable to majority of monomers subject to radical polymerization. The process can be used in the synthesis of well-defined homo-, gradient, diblock, triblock, and star polymers and more complex architectures, which include microgels and polymer brushes.

When preparing, for example, a block copolymer in the presence of the control agent, the end of the growing block is provided with a specific functionality that controls the growth of the block by means of reversible free radical deactivation. The functionality at the end of the block is of such a nature that it can reactivate the growth of the block in a second and/or third stage of the polymerization process with other ethylenically unsaturated monomers providing a covalent bond between, for example, a first and second block [A] and [B] and with any further optional blocks.

Further details on the chemistry of synthesis of block copolymers by RAFT processes may be found in the following publications, each of which is herein incorporated in its entirety by reference: Polymer, 2008, volume 49, 1079-1131; Chemical Society Reviews, 2014, volume 43, 496-505; Macromolecules, 1998, volume 31, 5559-5562; and Polymer, 2013, volume 54, 2011-2019.

In one non-limiting embodiment, the block copolymer according to the disclosed and/or claimed inventive concepts is obtained by RAFT-mediated controlled radical polymerization. In one non-limiting embodiment, the reversible transfer agents may be one or more compounds selected from the group consisting of dithioesters, thioether-thiones, trithiocarbonates, dithiocarbamates, xanthates and mixtures thereof.

In one non-limiting embodiment, the average degree of polymerization (DP) for block A of the block copolymer is a value ranging from about 5 to about 100,000. In another non-limiting embodiment, the average DP for the block A is a value ranging from about 5 to about 10,000. In yet another non-limiting embodiment, the average DP for the block A is a value ranging from about 10 to about 10,000. In yet another non-limiting embodiment, the average DP for the block A is a value ranging from about 10 to about 1000.

In one non-limiting embodiment, the average DP for block B is a value ranging from about 10 to about 100,000. In another non-limiting embodiment, the average DP for the block B is a value ranging from about 10 to about 10,000. In yet another non-limiting embodiment, the average DP for the block B is a value ranging from about 10 to about 1000.

The block copolymers according to the disclosed and/or claimed inventive concept(s) may be prepared according to the examples set out below. These examples are presented herein for purposes of illustration of the disclosed and/or claimed inventive concept(s) and are not intended to be limiting, for example, the preparations of the polymers. In the examples, the following abbreviations are used:
OAA: N-tert-octyl acrylamide
DMAC: N,N-dimethyl acrylamide
AIBN: 2,2′-azobis(2-methylpropionitrile)
DDMAT: 2-(dodecylthiocarbonothioylthio)-2-methylpropionic acid
EGDA: ethylene glycol diacrylate
PEGDA: polyethylene glycol diacrylate
CTA: Chain transfer agent
NMR: Nuclear magnetic resonance
UV: Ultraviolet
DLS: Dynamic light scattering
GPC: Gel permeation chromatography
TEM: Transmission electron microscopy
DSC: Differential Scanning Calorimetry
SEM: Scanning electron microscopy
PISA: Polymerization Induced Self Assembly
DP: Degree of polymerization
PMMA: Poly(methyl)methacrylate
Mₙ: Number-average molecular weight
M_{w}: Weight-average molecular weight

### EXAMPLES

### Example 1: Synthesis of poly(OAA)₈₅-poly(DMAC)₁₀₀ Diblock Copolymer

### Part A: Preparation of poly(OAA)₈₅ Homopolymer

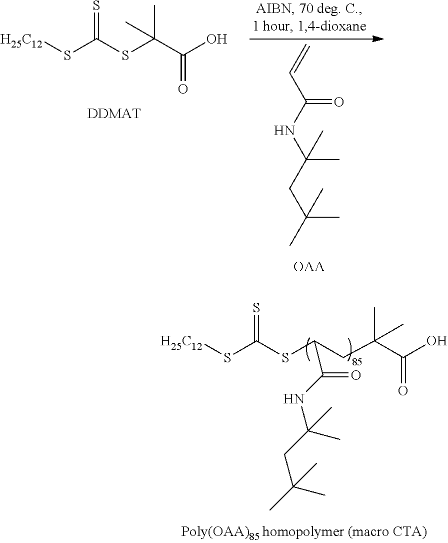

OAA (20.11 g, 0.11 mol), DDMAT RAFT agent (0.40 g, 1.10 mmol; target DP=100), AIBN (18.0 mg, 0.11 mmol; DDMAT/AIBN molar ratio=10) and 1,4-dioxane (30.79 g, 40% w/w) were weighed into a 100 mL round-bottom flask and degassed under N₂ with continuous magnetic stirring for 20 min. The OAA polymerization was allowed to proceed for 60 min in an oil bath set to 70° C., before quenching by exposing the hot reaction solution to air while cooling to 20° C. ¹H NMR spectroscopy studies indicated a final monomer conversion of 82%. The crude homopolymer was precipitated into excess methanol to remove residual OAA monomer before placing in a vacuum oven at 30° C. for three days to afford a dry yellow powder. The mean DP was calculated to be 85 by end-group analysis using UV spectroscopy (λ=308 nm). Chloroform GPC analysis indicated an Mₙ of 9900 g mol⁻¹ and an M_{w}/Mₙ of 1.18 using a series of ten near-monodisperse PMMA calibration standards.

### Part B: Preparation of poly(OAA)₈₅-poly(DMAC)₁₀₀ Diblock Copolymer

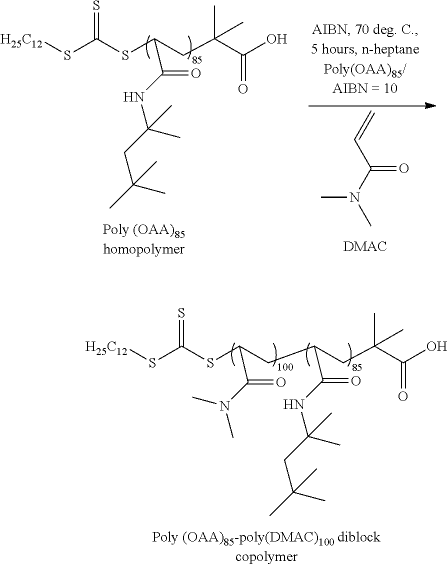

A typical protocol for the synthesis of poly(OAA)₈₅-poly(DMAC)₁₀₀ diblock copolymer nanoparticles in n-heptane was conducted as follows: poly(OAA)₈₅-macro-CTA (0.30 g, 18.8 μmol), DMAC (0.19 g, 1.88 mmol; target DP=100) and AIBN (0.30 mg, 1.88 μmol; 0.03 g of a 10 mg g⁻¹ stock solution of AIBN dissolved in DMAC; poly(OAA)₈₅/AIBN molar ratio=10) were dissolved in n-heptane (1.95 g; targeting 20% w/w solids). The reaction vial was sealed and degassed via N₂ gas for 15 min at 20° C. before being immersed in a pre-heated oil bath for 5 h at 70° C. The DMAC polymerization was quenched by exposing the hot reaction solution to air while cooling to 20° C. The resulting diblock copolymer nanoparticles were characterized by ¹H NMR spectroscopy in CDCl₃, with 0.1% w/w dispersions being prepared by dilution with n-heptane for DLS and TEM studies. Chloroform GPC analysis indicated an Mₙ of 19,900 g mol⁻¹ and an M_{w}/Mₙ of 1.19 (vs. a series of ten PMMA standards).

### Example 2: Synthesis of poly(OAA)₈₅-poly(DMAC)ₓ Diblock Copolymer

Consistent with the process described in Example 1, other diblock copolymer compositions (with targeted DP of 50, 75, 100, 125, 150, 175, 200, 225, and 250 for DMAC) were prepared in n-heptane by adjusting the amount of DMAC monomer to target the desired DP. For these additional syntheses, the volume of the continuous phase was adjusted to maintain an overall copolymer concentration of 20% w/w solids. ¹H NMR analysis indicated that at least 98% DMAC conversion was achieved in all cases. Poly(OAA)₈₅-poly(DMAC)ₓ diblock copolymer nanoparticles were also prepared in n-octane, n-decane, n-dodecane, n-tetradecane and n-hexadecane. All synthetic parameters except for the volume of solvent were unchanged. Owing to the differing densities of these n-alkanes, the overall solution volume varied for these formulations. Reasonably good RAFT control was achieved, although a gradual increase in M_{w}/Mₙ is discernible when targeting longer poly(DMAC) DPs.

### Example 3: One Pot Synthesis of poly(OAA)₄₁-poly(DMAC)₅₅ Diblock Copolymer

OAA (800 mg, 4.36 mmol), DDMAT RAFT agent (22.7 mg, 60 μmol; target DP=40) and AIBN (1.75 mg, 10 μmol; DDMAT/AIBN molar ratio=10) were dissolved in n-heptane (1.26 g; targeting 40% w/w solids) in a reaction vial. The resulting solution was then degassed for 20 min at 20° C. using a N₂ sparge before immersing the reaction vial in a pre-heated oil bath set at 70° C. After 150 min, ¹H NMR studies indicated 98% OAA conversion and a mean DP of 41. Chloroform GPC analysis indicated an Mₙ of 4,800 g mol⁻¹ and an M_{w}/Mₙ of 1.27. Next, deoxygenated n-heptane (4.60 mL; targeting 20% w/w solids) was added to dilute the reaction solution containing the poly(OAA)₄₁ macro-CTA. Then DMAC (0.62 g, 6.27 mmol; target DP=60) and AIBN (1.00 mg, 10 μmol; 0.10 g of a 10 mg g⁻¹ AIBN stock solution dissolved in DMAC; poly(OAA)₄₁/AIBN molar ratio 10) were combined and deoxygenated for 20 min at 20° C. before 0.66 mL of this monomer/initiator solution was added to the 20% w/w solution of poly(OAA)₄₁ macro-CTA in n-heptane. The DMAC polymerization was allowed to proceed for 5 h at 70° C., resulting in a dispersion of poly(OAA)₄₁-poly(DMAC)₅₅ diblock copolymer nanoparticles (Mₙ=13,500 g mol⁻¹ and M_{w}/Mₙ=1.24 by chloroform GPC analysis using PMMA calibration standards).

### Example 4: Synthesis of Core-Crosslinked poly(OAA)₈₅-poly(DMAC)₁₀₀-PEGDA₂₀ Triblock Copolymer Nanoparticles Via Sequential RAFT Dispersion Polymerization of DMAC and EGDA in n-heptane

A typical protocol for the synthesis of core-crosslinked poly(OAA)₈₅-poly(DMAC)₁₀₀-PEGDA₂₀ nanoparticles was conducted as follows: Poly(OAA)₈₅ macro-CTA (0.40 g, 25.1 μmol), DMAC (0.25 g, 2.51 mmol; target DP=100) and AIBN (0.40 mg, 2.51 μmol; 0.04 g of a 10 mg g⁻¹ stock solution of AIBN dissolved in DMAC; Poly(OAA)₈₅/AIBN molar ratio=10) were dissolved in n-heptane (2.94 g; targeting 20% w/w solids). The reaction vial was sealed and degassed under N₂ for 15 min at 20° C. before being placed in a pre-heated oil bath set at 70° C. for 195 min. EGDA (0.09 g, 0.50 mmol; target DP=20, previously degassed with N₂ gas at 20° C.) was then added using a deoxygenated syringe/needle. EGDA polymerization was allowed to proceed for 4 h before quenching by exposure of the hot reaction mixture to air while cooling to 20° C. The resulting cross-linked triblock copolymer nanoparticles were diluted with n-heptane to afford a 0.1% w/w dispersion prior to characterization by DLS and TEM.

### Characterization of Copolymers and Pickering Emulsions

¹H NMR spectroscopy: Spectra were recorded for poly(OAA)ₓ homopolymers and poly(OAA)₈₅-poly(DMAC)ₓ diblock copolymers dissolved in CDCl₃ using a 400 MHz Bruker Avance 400 spectrometer with 64 scans being averaged per spectrum.

UV spectroscopy: UV absorption spectra were recorded between 200 and 800 nm using a PC-controlled UV-1800 spectrophotometer at 25° C. equipped with a 1 cm path length cell. A Beer-Lambert curve was constructed using a series of fourteen DDMAT solutions of known concentration in chloroform. The absorption maximum at 308 nm assigned to the trithiocarbonate end-group was used for this calibration plot, and DDMAT concentrations were selected such that the absorbance at this wavelength always remained below unity. Subsequently, the mean DP for each of the five poly(OAA) homopolymers was determined using the molar extinction coefficient (E) determined for DDMAT alone, for which ε=15,210±170 mol⁻¹ dm³ cm⁻¹.

GPC: Molecular weight data for the five poly(OAA)ₓ homopolymer precursors and the corresponding series of poly(OAA)₈₅-poly(DMAC)ₓ diblock copolymers were obtained using chloroform GPC at 35° C., with the eluent containing 0.25% TEA by volume. Two Polymer Laboratories PL gel 5 μm Mixed C columns were connected in series to a Varian 390 multidetector suite (only the refractive index detector was used) and a Varian 290 LC pump injection module at a flow rate of 1.0 mL min⁻¹. Ten near-monodisperse PMMA standards (Mₙ=625-618,000 g mol⁻¹) were used for calibration and data were analyzed using Varian Cirrus GPC software supplied by the instrument manufacturer.

DLS: A Malvern Zetasizer NanoZS instrument was used to determine the intensity-average hydrodynamic diameter of the copolymer nanoparticles at 20° C. at a fixed scattering angle of 173°. As-synthesized dispersions were diluted to 0.1% w/w using n-heptane and analyzed using a 1.0 cm path length glass cuvette. Data were averaged over three consecutive measurements (with 10 sub-runs per run) for each sample. Sphere-equivalent intensity-average diameters were calculated for nanoparticles using the Stokes-Einstein equation, which assumes perfectly monodisperse, non-interacting spheres.

TEM: Copper/palladium grids were surface-coated in-house to produce a thin film of amorphous carbon. A 15 μL droplet of a 0.1% w/w copolymer dispersion (prepared by serial dilution using n-heptane) was placed on a grid using a micropipette, allowed to dry, and then stained by exposed to ruthenium(IV) oxide vapor for 7 min at 20° C. prior to analysis. A FEI Tecnai Spirit microscope operating at 80 kV and equipped with a Gatan 1kMS600CW CCD camera was used to image the nanoparticles.

DSC: Glass transition temperatures (T_{g}) for the five poly(OAA)ₓ homopolymers were determined using a TA Instruments Discovery DSC 25 instrument operating from −50° C. to 120° C. at a heating/cooling rate of 10° C. min⁻¹. Each homopolymer (10 mg) was dried for at least 24 h in a vacuum oven at 30° C. prior to analysis. Dried samples were hermetically sealed in a vented aluminum pan, and the instrument was calibrated for heat flow and temperature using both indium and zinc standards. Samples were annealed at 100° C. for 5 min before cooling to −50° C., with this latter temperature being maintained for 1 min. The T_{g} was then determined by heating the homopolymer up to 120° C. and determining the mid-point value. Heat flow was also monitored for n-dodecane alone, a 20% w/w solution of a poly(OAA)₈₅ homopolymer in n-dodecane and 20% w/w dispersions of poly(OAA)₈₅-poly(DMAC)₁₅₀ diblock copolymer nanoparticles in n-dodecane on cooling from 120° C. to −50° C. at 10° C. min⁻¹.

Turbidimetry: These experiments were undertaken for poly(OAA)₈₅-poly(DMAC)₁₀₀ diblock copolymer nanoparticles prepared directly in various n-alkanes. The corresponding n-alkane was used as a diluent to afford a 1.0% w/w dispersion in each case. A Varian Cary 300 Bio UV-visible spectrometer was used to record transmittance vs. temperature plots at a fixed wavelength of 600 nm. Each 1.0% w/w dispersion was equilibrated for 5 min at 90° C. and then cooled to either 20 or 2° C. at a rate of 1.0° C. per min with the transmittance being recorded at 1.0° C. intervals.

### Pickering Emulsions

The performance of poly(OAA)₈₅-poly(DMAC)ₓ nanoparticles as putative Pickering emulsifiers was briefly investigated. Accordingly, poly(OAA)₈₅-poly(DMAC)₁₅₀ nanoparticles were prepared on a three-gram scale in n-heptane to provide sufficient material for a series of experiments. This solvent was selected because we wanted to ensure a high degree of dispersion for the nanoparticles at ambient temperature. The nanoparticle concentration was systematically lowered from 1.0% w/w to 0.075% w/w prior to addition of deionized water and high shear homogenization at a constant n-heptane volume fraction of 0.50. The electrical conductivity for an emulsion obtained using 1.0% w/w poly(OAA)₈₅-poly(DMAC)₁₅₀ copolymer was determined to be 1.85×10⁻⁴ S m⁻¹, which is comparable to that of deionized water alone (1.77×10⁻⁴ S m⁻¹). This indicates the formation of an o/w emulsion. The ‘drop test’ method (which involves taking an aliquot of the emulsion and determining whether it disperses more readily when added to either water or n-heptane) was used to confirm that o/w emulsions were always produced regardless of the nanoparticle concentration.

Laser diffraction was used to size these emulsions. At higher nanoparticle concentrations, the volume-average droplet diameter remained constant at around 25-30 μm. However, both the mean droplet diameter and the standard deviation increased at nanoparticle concentrations were reduced below 0.25% w/w.

What we claim is:

1. A block copolymer comprising:
at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each said alkyl moiety independently has from 1 to 3 carbons; and
at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each said alkyl moiety independently has from 4 to about 40 carbons.

2. The block copolymer according to claim 1 comprising:
at least one block A comprising repeating units derived from at least one monomer selected from the group consisting of functionalized or unfunctionalized acylamide, N-methyl acrylamide, N,N′-dimethyl acrylamide, (meth)acylamide, N-methyl (meth)acrylamide, N,N′-dimethyl (meth)acrylamide, and combinations thereof; and
at least one block B comprising repeating units derived from at least one monomer selected from the group consisting of functionalized or unfunctionalized N-butyl acrylamide, N-sec-butyl acrylamide, N-tert-butyl acrylamide, N-isobutyl acrylamide, N-neopentyl acrylamide, N-hexyl acrylamide, N-cyclohexyl acrylamide, N-2-ethylbutyl acrylamide, N-heptyl acrylamide, N-2-heptyl acrylamide, N-2-ethylhexyl acrylamide, N-octyl acrylamide, N-tert-octyl acrylamide, N-iso-decyl acrylamide, N-dodecyl acrylamide, N-lauryl acrylamide, N-2-propylheptyl acrylamide, N-behenyl acrylamide, N-tetradecyl acrylamide, N-octadecyl acrylamide, N-butyl (meth)acrylamide, N-sec-butyl (meth)acrylamide, N-tert-butyl (meth)acrylamide, N-isobutyl (meth)acrylamide, N-neopentyl (meth)acrylamide, N-hexyl (meth)acrylamide, N-cyclohexyl (meth)acrylamide, N-2-ethylbutyl (meth)acrylamide, N-heptyl (meth)acrylamide, N-2-heptyl (meth)acrylamide, N-2-ethylhexyl (meth)acrylamide, N-octyl (meth)acrylamide, N-tert-octyl (meth)acrylamide, N-iso-decyl (meth)acrylamide, N-dodecyl (meth)acrylamide, N-lauryl (meth)acrylamide, N-2-propylheptyl (meth)acrylamide, N-behenyl (meth)acrylamide, N-tetradecyl (meth)acrylamide, N-octadecyl (meth)acrylamide, and combinations thereof.

3. A crosslinked block copolymer comprising:
at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each said alkyl moiety independently has from 1 to 3 carbons;
at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each said alkyl moiety independently has from 4 to about 40 carbons; and
repeating units derived from at least one crosslinking agent.

4. The block copolymer according to claim 1 or claim 2 that is a diblock, triblock, or multiblock copolymer.

5. The crosslinked block copolymer according to claim 3 that is a crosslinked diblock, triblock, or multiblock copolymer.

6. The crosslinked block copolymer according to claim 3 wherein said crosslinking agent is selected from the group consisting of allyl acrylate, ethylene glycol acrylate, ethylene glycol diacrylate, diethylene glycol acrylate, diethylene glycol diacrylate, triethylene glycol acrylate, triethylene glycol diacrylate, polyethylene glycol acrylate, polyethylene glycol diacrylate, butylene glycol acrylate, butylene glycol diacrylate, glycidyl acrylate, N-hydroxymethyl acrylamide, N,N′-methylene diacrylamide, trimethylolpropane triacrylate, allyl methacrylate, ethylene glycol methacrylate, ethylene glycol dimethacrylate, diethylene glycol methacrylate, diethylene glycol dimethacrylate, triethylene glycol methacrylate, triethylene glycol dimethacrylate, polyethylene glycol methacrylate, polyethylene glycol dimethacrylate, butylene glycol methacrylate, butylene glycol dimethacrylate, glycidyl methacrylate, N-hydroxymethyl methacrylamide, N,N′-methylene dimethacrylamide, trimethylolpropane trimethacrylate, divinylbenzene, triallyl isocyanurate, triallyl citrate, diethyleneglycol divinyl ether, pentaerythritol triallyl ether, pentaerythritol triacrylate, pentaerytbritol trimethacrylate, pentaerythritol tetraacrylate, pentaerythritol tetramethacrylate, methylene bisacrylamide, methylene bismethacrylamide, N,N′-divinylimidazolidone, triallyl-1,3,5-triazine-2,4,6(1H,3H,5H)-trione, 2,4,6-triallyloxy-1,3,5-triazine, and combinations thereof.

7. The block copolymer according to claim 2 having a structure selected from the group consisting of: 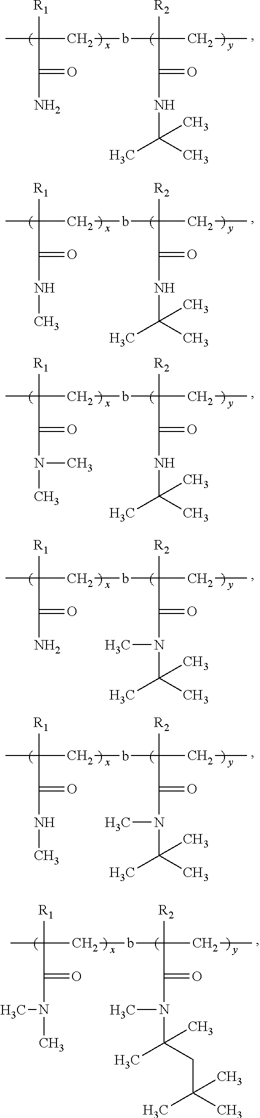 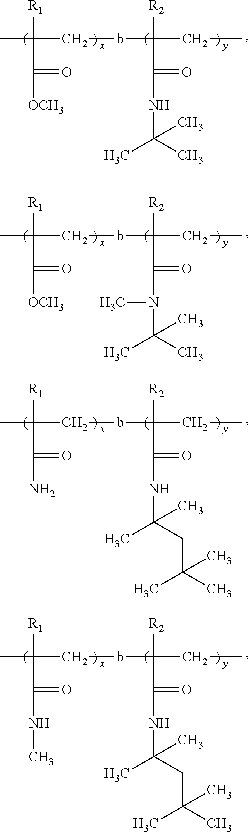 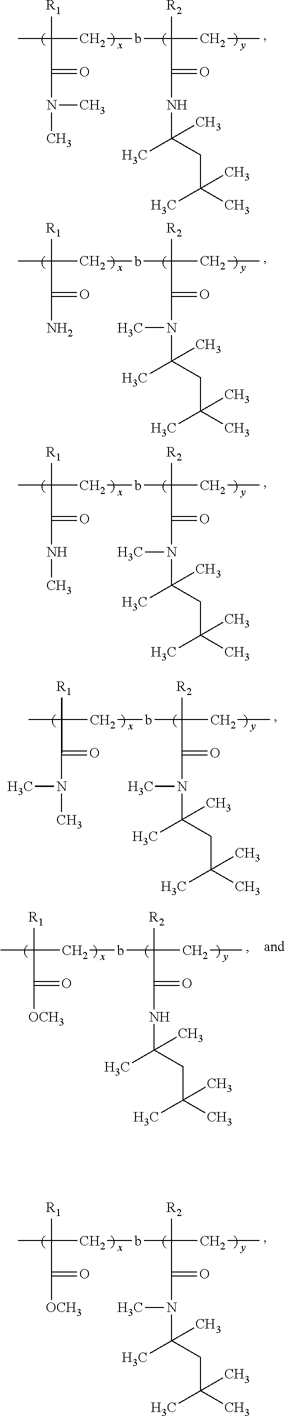
wherein each x and y is independently an integer having a value from about 10 to about 10000, and each R₁ and R₂ is independently selected from the group consisting of hydrogen, methyl, and combinations thereof.

8. A composition comprising a block copolymer comprising:
at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each said alkyl moiety independently has from 1 to 3 carbons;
at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each said alkyl moiety independently has from 4 to about 40 carbons; and
optionally, repeating units derived from at least one crosslinking agent.

9. The composition according to claim 8 that is a personal care composition, pharmaceutical composition, coating composition, construction composition, nutritional composition, agricultural composition, adhesive composition, oilfield composition, household, industrial and institutional composition, cementing fluid, servicing fluid, gravel packing mud, fracturing fluid, completion fluid, work-over fluid, spacer fluid, drilling mud, biocide, ink, paper, polish, membrane, metal working fluid, plastic, textile, printing composition, lubricant, detergent, battery composition, glass coating composition, or preservative composition.

10. A personal care composition comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each said alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each said alkyl moiety independently has from 4 to about 40 carbons; and optionally, repeating units derived from at least one crosslinking agent.

11. The personal care composition according to claim 10 that is a sun care composition, a face care composition, a lip care composition, an eye care composition, a skin care composition, an after-sun composition, a body care composition, a nail care composition, an anti-aging composition, an insect repellant, an oral care composition, a deodorant composition, a hair care composition, a conditioning composition, a color cosmetic composition, a color-protection composition, a self-tanning composition, or a foot care composition.

12. The personal care composition according to claim 11 further comprising at least one additive selected from the group consisting of UV actives, UV active solubilizers, oils, waxes, solvents, emulsifiers, preservatives, antioxidants, antiradical protecting agents, vitamins, perfumes, insect repellants, dyes, pigments, humectants, fillers, thickeners, film formers, stabilizers, buffers, spreading agents, pearlizing agents, electrolytes, acids, bases, crystalline structuring agents, abrasives, pharmaceutically or cosmetically acceptable excipients, and combinations thereof.

13. A composition in the form of colloidal particles comprising a block copolymer wherein said copolymer comprises at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each said alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each said alkyl moiety independently has from 4 to about 40 carbons; and optionally, repeating units derived from at least one crosslinking agent.

14. The composition according to claim 13 wherein said colloidal particles have a mean diameter ranging from about 10 to about 1000 nanometer.

15. A Pickering emulsion composition comprising a block copolymer comprising at least one block A comprising repeating units derived from at least one monomer selected from the group consisting of functionalized or unfunctionalized acylamides, N-alkyl acrylamides, N-alkyl,N′-alkyl acrylamides, (meth)acrylamides, N-alkyl (meth)acrylamides, N-alkyl,N′-alkyl (meth)acrylamides, acrylates, alkyl acrylates, (meth)acrylates, alkyl (meth)acrylates, and combinations thereof, wherein each said alkyl moiety independently has from 1 to 3 carbons; at least one block B comprising repeating units derived from at least one monomer selected from the group consisting of functionalized or unfunctionalized N-alkyl acrylamides, N-alkyl,N′-alkyl acrylamides, N-alkyl (meth)acrylamides, N-alkyl,N′-alkyl (meth)acrylamides, and combinations thereof, wherein each said alkyl moiety independently has from 4 to about 40 carbons; and optionally, repeating units derived from at least one crosslinking agent.

16. The Pickering emulsion according to claim 15 wherein said block copolymer comprises at least one block A comprising repeating units derived from at least one monomer selected from the group consisting of functionalized or unfunctionalized acylamide, N-methyl acrylamide, N,N′-dimethyl acrylamide, (meth)acylamide, N-methyl (meth)acrylamide, N,N′-dimethyl (meth)acrylamide, and combinations thereof; and at least one block B comprising repeating units derived from at least one monomer selected from the group consisting of functionalized or unfunctionalized N-butyl acrylamide, N-sec-butyl acrylamide, N-tert-butyl acrylamide, N-isobutyl acrylamide, N-neopentyl acrylamide, N-hexyl acrylamide, N-cyclohexyl acrylamide, N-2-ethylbutyl acrylamide, N-heptyl acrylamide, N-2-heptyl acrylamide, N-2-ethylhexyl acrylamide, N-octyl acrylamide, N-tert-octyl acrylamide, N-iso-decyl acrylamide, N-dodecyl acrylamide, N-lauryl acrylamide, N-2-propylheptyl acrylamide, N-behenyl acrylamide, V-tetradecyl acrylamide, N-octadecyl acrylamide, N-butyl (meth)acrylamide, N-sec-butyl (meth)acrylamide, N-tert-butyl (meth)acrylamide, N-isobutyl (meth)acrylamide, N-neopentyl (meth)acrylamide, N-hexyl (meth)acrylamide, N-cyclohexyl (meth)acrylamide, N-2-ethylbutyl (meth)acrylamide, N-heptyl (meth)acrylamide, N-2-heptyl (meth)acrylamide, N-2-ethylhexyl (meth)acrylamide, N-octyl (meth)acrylamide, N-tert-octyl (meth)acrylamide, N-iso-decyl (meth)acrylamide, N-dodecyl (meth)acrylamide, N-lauryl (meth)acrylamide, N-2-propylheptyl (meth)acrylamide, N-behenyl (meth)acrylamide, N-tetradecyl (meth)acrylamide, N-octadecyl (meth)acrylamide, and combinations thereof.
